SEQUENCE LISTING (1 sequence)

SEQ ID NO: 1 - DNA, 1498 bp, Pseudomonas sp.
agagtttgatcctggctcagattgaacgctggcggcaggcctaacacatgcaagtcgagcggatgaggggtgcttgcactctgattcagcggcggacgggtgagtaatgcctaggaatctgcccgatagtgggggacaacgtttcgaaaggaacgctaataccgcatacgtcctacgggagaaagtgggggatcttcggacctcacgctatcggatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggctgtcggctaataccctgcagttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaagccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagctgttgggttccttgagaacttagtagcgaagctaacgcgataagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacagagggttgccaagccgcgaggcggagctaatctcacaaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaacaaggtaacc

(19) United States
(12) Patent Application Publication
(10) Pub. No.: US 2021/0269341 A1
(43) Pub. Date: Sep. 2, 2021

(54) LAS-DEGRADING AND/OR N-REMOVING BACTERIUM AND APPLICATION THEREOF

(71) Applicants: Agro-Environmental Protection Institute, Ministry of Agriculture and Rural Affairs, Tianjin (CN); Tianjin Agricultural University, Tianjin (CN)

(72) Inventors: Peizhen CHEN, Tianjin (CN); Xiangqiun ZHENG, Tianjin (CN); Shaopeng Li, Tianjin (CN)

(73) Assignees: Agro-Environmental Protection Institute, Ministry of Agriculture and Rural Affairs, Tianjin (CN); Tianjin Agricultural University, Tianjin (CN)

(21) Appl. No.: 17/253,587

(22) PCT Filed: Jun. 4, 2019
(86) PCT No.: PCT/CN2019/089949

§ 371 Date: Dec. 17, 2020

(30) Foreign Application Priority Data
Jan. 15, 2019 (CN) 201910036228.9

(51) Int. Cl. C02F 3/34; C12N 1/20

(52) U.S. Cl. CPC C02F 3/34; C12R 2001/38; C12N 1/205

(57) ABSTRACT

A bacterium that degrades LAS and/or removes N is provided. The bacterium is taxonomically classified as Pseudomonas sp., and was deposited in China General Microbiological Culture Collection Center on Sep. 18, 2018, with the accession number of CGMCC 16502. The bacterium can be applied in the rural domestic sewage treatment, and has the effect of removing LAS and/or N pollution.

### FIELD OF THE INVENTION

The present invention belongs to the technical field of bioremediation, and particularly relates to a LAS-degrading and/or N-removing bacterium and applications thereof.

### BACKGROUND OF THE INVENTION

With the rapid improvement of the living conditions of rural residents in China, the discharge of rural domestic sewage has also been increasing year by year, and nitrogen (N) is one of the main pollutants in domestic sewage. Studies have shown that among the main pollutants which cause eutrophication of Lake Taihu, 25% of nitrogen and 60% of phosphorus are derived from rural domestic sewage. Although nitrogen is one of the nutrients largely required by plant growth, a significant increase in nitrogen in water would lead to a range of environmental problems, such as consumption of dissolved oxygen in water, production of cancerogenic nitrosamines, and eutrophication of water, posing a huge threat to the ecological environment and human health. Therefore, removal of nitrogen from polluted water is of great significance for protecting the environment, developing the economy and ensuring health. Linear alkylbenzene sulfonates (LAS) are a class of anionic surfactants widely used as household detergents and cleaning agents. According to statistics, in China detergents increased from about 1 million tons in 1985 to about 4.6 million tons in 2005. A large amount of LAS discharged into waterbody would not only result in production of foam that affects the natural landscape of water, but also have a significant influence on the redox capacity and enzymatic activity of microorganisms, which reduces the activity of the microorganisms and causes them to lose some of their original functions in the environment. Until now LAS have become another important organic pollutant in domestic sewage, and it is highly important to remove LAS and nitrogen from rural domestic sewage. Therefore, there is still a demand for bacteria that can be used for rural sewage treatment.

### SUMMARY OF THE INVENTION

It is an object of the present invention to provide a bacterium which degrades LAS (linear alkylbenzene sulfonates) and/or removes N (nitrogen). The bacterium was isolated and screened from the sewage of the rural septic tank in Ankang, Shaanxi (China), and can be used for sewage treatment.

The LAS-degrading and/or N-removing bacterium provided by the present invention is taxonomically classified as Pseudomonas sp., and was deposited in China General Microbiological Culture Collection Center (CGMCC; address: No. 3, Yard No. 1, West Beichen Road, Chaoyang District, Beijing, China) on Sep. 18, 2018, with the accession number of CGMCC 16502.

Preferably, the 16S rDNA sequence of the strain of the bacterium is represented by SEQ ID NO: 1, having a length of 1498 bp.

Preferably, the strain of the bacterium has the following phenotypic characteristics: when cultured at 36° C. for 24 h, the bacterial colonies are light yellow, circular, and opaque, and have a wet surface and a smooth margin; the bacteria cells are rod-shaped, 0.4˜0.8 μm×1.8˜4.7 μm in size, arranged individually or in pairs, and grain-negative.

Preferably, the strain of the bacterium is characterized by a (G+C)mol % of 63.4%.

Preferably, the strain of the bacterium has the following characteristics: the quinone components are ubiquinone Q8 and ubiquinone Q9, and their relative contents are 57.24% and 42.76%, respectively.

Preferably, the strain of the bacterium has the following characteristics: the major fatty acid components (relative content>10%) are: C_{16:0}, Summed Feature 3 (C_{16:1}ω7c or C_{16:1}ω6c), and Summed Feature 8 (C_{18:1}ω7c or C_{18:1}ω6c), and their relative contents are 31.16%, 14.85%, and 12.32%, respectively.

Preferably, the strain of the bacterium has the following characteristics: the cell wall-characteristic DAP component is meso-DPA; and the cell wall-characteristic sugar component is ribose.

Preferably, the strain of the bacterium has the following characteristics: the bacterial polar lipids comprise diphosphatidylglycerol, phosphatidylglycerol, phosphatidylethanolamine, phosphatidylcholine, an unidentified phospholipid, an unidentified lipid, and an unidentified aminophospholipid.

Preferably, the hybridization homology of the bacterial strain with the model strain Pseudomonas guangdongensis CCTCC AB2012022^{T} is 35.4%.

Preferably, the bacterium shows its LAS-degrading effect over an initial LAS concentration of 0-400 mg/L; and the bacterium shows its ammoniacal nitrogen-removing effect over an initial ammoniacal nitrogen concentration of 0-200 mg/L.

More preferably, the bacterium shows its LAS-degrading effect over an initial LAS concentration of 0-200 mg/L; and the bacterium shows its ammoniacal nitrogen-removing effect over an initial ammoniacal nitrogen concentration of 0-100 mg/L.

Even more preferably, the bacterium can simultaneously degrade LAS and remove N.

According to the morphological characteristics and the physiological and biochemical characteristics of the bacterium and the results of aligning its 16S rDNA to Genbank, a phylogenetic tree was constructed and the strain was comprehensively identified as Pseudomonas sp.

In particular, the sewage is rural domestic sewage.

In addition, the present invention provides a use of the bacterium for decontamination of rural domestic sewage, wherein the rural domestic sewage has a LAS concentration of 0 to 400 mg/L and/or an ammoniacal nitrogen concentration of 0 to 200 mg/L.

The LAS-degrading and/or N-removing bacterium according to the present invention has the following beneficial effects as compared with the prior art.

Under aerobic conditions, the bacterium of the present invention can assimilate LAS as the sole carbon source and ammoniacal nitrogen as the sole nitrogen source. By this characteristic of the strain, it can be directly applied to the rural domestic sewage containing LAS, such as the kitchen, bath sewage, etc., and the removal of N from the sewage with a high LAS content can be realized. As a result, it would have good application prospects and social benefits.

The LAS-degrading and/or N-removing bacterium according to the present invention is an indigenous microorganism screened from rural domestic sewage with a high salt content, and their application to the rural domestic sewage with a high salt content will achieve a considerable economic benefit, greatly save the cost for sewage treatment, and have no safety concern about the strain, thereby being suitable for use in rural areas of China where the gathering places are scattered. The bacterial strain according to the present invention has an excellent effect of degrading LAS-N composite pollutants, and addition of the bacteria can effectively improve the efficiency of the indigenous microorganism in removing N and LAS from rural domestic sewage.

It can be understood that, in the present application, “LAS-degrading and/or N-removing bacterium”, “bacterium which degrades LAS and/or removes N”, “Pseudomonas” and “Pseudomonas sp.” have the same meaning, and “bacterium”, “species” and “strain” have the same meaning in some places. LAS refers to linear alkylbenzene sulfonate. Both N and “nitrogen” refer to the nitrogen element.

### Deposition Information

Bacterium name: LAS-degrading and/or N-removing bacterium

Taxonomy: Pseudomonas sp.

Strain No.: A-1

Depository: China General Microbiological Culture Collection Center

Depository abbreviation: CGMCC

Address of Depository: No. 3, Yard No. 1, West Beichen Road, Chaoyang District, Beijing, China

Date of deposit: Sep. 18, 2018

Depository Accession Number: CGMCC 16502

### BRIEF DESCRIPTION OF THE DRAWINGS

FIG. 1 is an electron micrograph showing the morphology of cells of the LAS-degrading and/or N-removing bacterium according to the present invention.

FIG. 2 is a photograph showing the morphology of colonies of the LAS-degrading and/or N-removing bacterium according to the present invention.

FIG. 3 is a 16S rDNA sequence genetic tree of the LAS-degrading and/or N-removing bacterium according to the present invention and related species.

FIG. 4 is a diagram showing the nitrogen removing effect of the LAS-degrading and/or N-removing bacterium according to the present invention at different initial concentrations of ammoniacal nitrogen.

### DETAILED DESCRIPTION OF THE INVENTION

### Example 1 Isolation and Identification of the Bacterial Strain of the Present Invention

(1) Medium

\textcircled{1} Medium for nitrogen removal: 0.48 g of ammonium sulfate, 4.71 g of succinic acid and a 50 mL trace element solution were dissolved and then made to a volume of 1 L with distilled water, with the pH controlled between 7.0 and 7.2. (for a solid medium, 2% agar was added). The trace element solution: 6.5 g of K₂HPO₄.3H₂O, 2.5 g of MgSO₄.7H₂O, 2.5 g of NaCl, 0.05 g of FeSO₄.7H₂O, and 0.04 g of MnSO₄.H₂O were dissolved and made to a volume of 1 L with distilled water.

\textcircled{2} LAS-degrading selective medium: 0.48 g of ammonium sulfate, 4.71 g of succinic acid, 0.10 g of LAS, and a 50 mL trace element solution. The trace element solution: 6.5 g of K₂HPO₄.3H₂O, 2.5 g of MgSO₄.7H₂O, 2.5 g of NaCl, 0.05 g of FeSO₄.7H₂O, and 0.04 g of MnSO₄.H₂O, which were dissolved and made to a volume of 1 L with distilled water.

(2) Isolation and screening of nitrogen removing strains

10 mL of sewage from the septic tank in Ankang, Shaanxi was taken with a sterile pipette, placed in a 250 ml Erlenmeyer flask (containing 100 mL sterile medium for nitrogen removal), and cultured at 30° C. on a shaker at 160 rpm. Each passage was cultured for three days. The presence of nitrobacteria was detected by using a diphenylamine reagent. The bacterial liquid was cultured until the presence of nitrobacteria was detected in the medium.

The culture liquid containing the nitrobacteria was plated by limiting dilution, and cultured in an incubator at 37° C. for 48 hours. Afterwards single colonies were picked, and inoculated by the streak plate method for multiple times. When no contaminating bacteria were observed under a microscope, the purification of the strain was completed.

The purified strain was subjected to a screening experiment for ammoniacal nitrogen removal. The strains were individually inoculated in the medium for nitrogen removal, and cultured in an incubator at 37° C. for 48 hours. The ammoniacal nitrogen removal was determined, and the strains with the ammoniacal nitrogen removal higher than 85% were selected as the nitrogen-removing strains.

(3) Screening for nitrogen-removing and LAS-degrading strains

The selected nitrogen-removing strains were inoculated into the liquid LAS-degrading selective medium in an inoculation amount of 10%, and cultured on a shaker for 48 hours, and the degradation of LAS was measured. A strain showing a degradation higher than 80% is a LAS-degrading and/or N-removing bacterium.

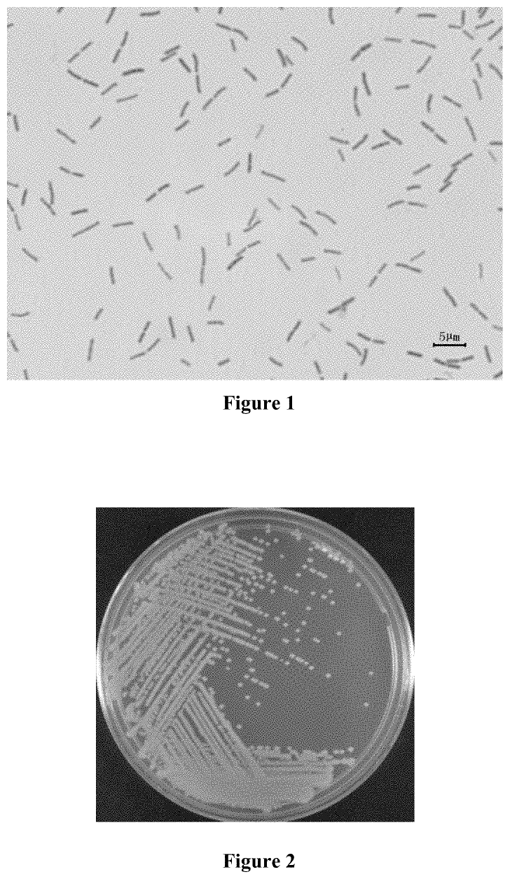

(4) A LAS-degrading and/or N-removing bacterium was identified by screening. FIG. 1 is an electron micrograph showing the morphology of cells of the LAS-degrading and/or N-removing bacterium according to the present invention. FIG. 2 is a photograph showing the morphology of colonies of the LAS-degrading and/or N-removing bacterium according to the present invention. It can be seen from FIG. 1 and FIG. 2 that the strain of bacterium has the following phenotypic characteristics: when cultured at 36° C. for 24 h, the bacterial colonies are light yellow, circular, and opaque, and have a wet surface and a smooth margin; the bacteria cells are rod-shaped, 0.4˜0.8 μm×1.8˜4.7 μm in size, arranged individually or in pairs, and grain-negative.

Furthermore, the bacterial strain was assayed and has the following characteristics: (G+C)mol % is 63.4%; the quinone components are ubiquinone Q8 and ubiquinone Q9, and their relative contents are 57.24% and 42.76%, respectively; the major fatty acid components (relative content>10%) are: C_{16:0}, Summed Feature 3 (C_{16:1}ω7c or C_{16:1}ω6c), and Summed Feature 8 (C_{18:1}ω7c or C_{18:1}ω6c), and their relative contents are 31.16%, 14.85% and 12.32%, respectively; the cell wall-characteristic DAP component is meso-DPA; and the cell wall-characteristic sugar component is ribose; the polar lipid of this strain comprises diphosphatidylglycerol, phosphatidylglycerol, phosphatidylethanolamine, phosphatidylcholine, an unidentified phospholipid, an unidentified lipid, and an unidentified aminophospholipid; and the hybridization homology of the bacterial strain with the model strain Pseudomonas guangdongensis CCTCC AB2012022^{T} is 35.4%.

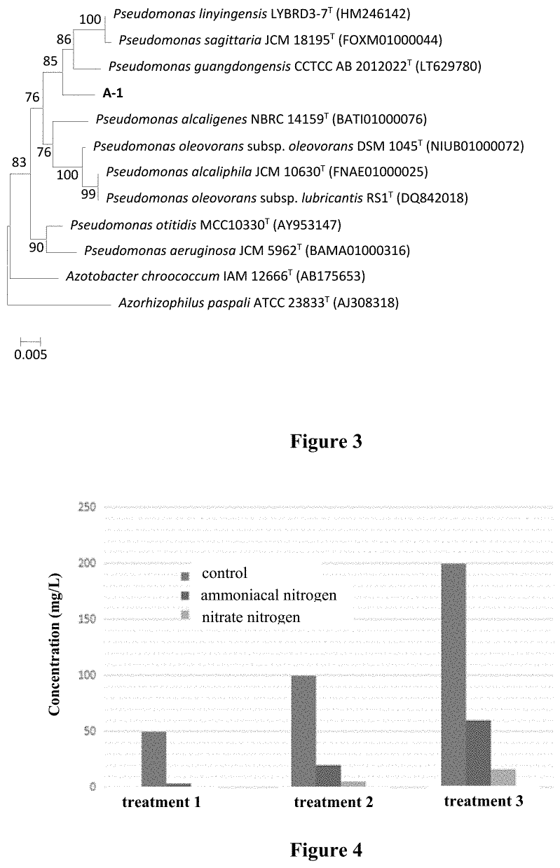

(5) 16S rDNA sequence analysis and phylogenetic analysis The 16S rDNA gene sequence was 1498 bp in length as confirmed by PCR amplification and sequencing of the 16S rDNA (see SEQ ID NO: 1). Through the alignment by submission to Genbank, it was found that the LAS-degrading and/or N-removing bacterium according to the present invention can have a homology as high as 97.88% with Pseudomonas linyingensis LYBRD3-7^{T}. The 16S rDNA sequence genetic tree of the strain and some related species was generated with the software MEGA 5.0 by the neighbor-joining method, and the similarity was repeatedly calculated for 1,000 times. In FIG. 3, the genetic tree nodes only showed those with Bootstrap values greater than 50% (see FIG. 3; FIG. 3 is a 16S rDNA sequence genetic tree of the strain of the present invention and related species).

(6) The physiological and biochemical results of the LAS-degrading and/or N-removing bacterium according to the present invention are shown in Table 2 below.

TABLE 2
Physiological and Biochemical Characteristics:

| | | | | | | | |
|---|---|---|---|---|---|---|---|
| Ala-Phe-Pro | − | adonitol | − | L-pyrrolidonyl | − | L-Arabitol | − |
| arylamidase | | | | arylamidase | | | |
| D-cellobiose | − | β-galactosidase | − | H₂S Production | − | β-N-acetylamino- | − |
| | | | | | | glucosidase | |
| Glutamyl | − | D-glucose | − | γ-glutamyl- | − | fermentation/glucose | − |
| arylamidase | | | | transferase | | | |
| pNA | | | | | | | |
| β-Glucosidase | − | D-maltose | + | D-mannitol | − | D-mannose | − |
| β-xylosidase | − | β-Alanine | − | L-proline | + | lipase | − |
| | | arylamidase pNA | | arylamidase | | | |
| palatinose | − | tyrosine | + | urease | − | D-sorbitol | − |
| | | arylamidase | | | | | |
| sucrose | − | D-tagatose | − | D-trehalose | − | Citrate (sodium) | − |
| malonate | − | 5-keto-D-Gluconate | − | L-lactate | − | α-glucosidase | − |
| | | | | alkalinisation | | | |
| succinate | − | β-N-Acetyl- | − | α-galactosidase | − | phosphatase | − |
| alkalinisation | | galactosaminidase | | | | | |
| Glycine | − | ornithine | − | lysine | − | L-Histidine | − |
| arylaminase | | decarboxylase | | decarboxylase | | assimilation | |
| coumaric acid | − | β-glucuronidase | − | O/129 | + | Glu-Gly-Arg- | − |
| | | | | resistance | | arylamidase | |
| L-malate | + | ELLMAN | − | L-lactate | − | | |
| assimilation | | | | assimilation | | | |

Symbol Description: “+”, positive; “−”, negative.

### Example 2 LAS-Degrading Test

The activated LAS-degrading and/or N-removing bacterium of the present invention was respectively inoculated into the media having LAS concentrations of 50 mg/L, 100 mg/L, 200 mg/L and 400 mg/L by a dilution coating method, with an inoculum amount of 1%. One blank control group and 3 parallel test groups were set up. After culturing under shaking for 48 hours, the degradation of LAS was determined. The results are shown in Table 3 below.

TABLE 3
Degradation of the strains of the present invention at different initial
concentrations of LAS

| | | | | | |
|---|---|---|---|---|---|
| Initial concentration | 0 | 50 | 100 | 200 | 400 |
| (mg/L) | | | | | |
| Removal of LAS | — | 87 | 82 | 78 | 50 |
| (%) | | | | | |

It can be seen from the results in Table 3 that the LAS-degrading and/or N-removing bacterium of the present invention can degrade LAS, and can exert this LAS-degrading function at an initial LAS concentration from 0 to 400 mg/L. In particular, the degradation of LAS was higher at the initial LAS concentration from 0 to 200 mg/L.

### Example 3 Nitrogen Removing Effect Experiment

The LAS-degrading and/or N-removing bacterial strain of the present invention was inoculated into 100 mL media for nitrogen removal at different nitrogen levels, with initial ammoniacal nitrogen concentrations of 50 mg/L (Treatment 1), 100 mg/L (Treatment 2) and 200 mg/L (Treatment 3), respectively. A medium not inoculated with the strain was used as a blank control. The media were cultured in an incubator at 37° C. under shaking at 160 rpm for 30 h. The culture solution was centrifuged at 10,000 rpm for 6 minutes, and filtered through a 0.45 μm filter. The contents of ammoniacal nitrogen and nitrate nitrogen in the culture solution were measured. The detailed measurement results are shown in FIG. 4. FIG. 4 is a diagram showing the nitrogen removing effect of the LAS-degrading and/or N-removing bacterial strain of the present invention at different initial ammoniacal nitrogen concentrations.

It can be seen from FIG. 4 that the LAS-degrading and/or N-removing bacterial strain of the present invention showed removals of 92%, 80% and 70% at different initial ammoniacal nitrogen concentrations, respectively, indicating that the LAS-degrading and/or N-removing bacterium of the present invention had a good nitrogen removing function, and can always exert this nitrogen removing function at an initial ammoniacal nitrogen concentration less than 200 mg/L, where the nitrogen removing effect was even better when the initial ammoniacal nitrogen concentration was less than 100 mg/L.

### Example 4 Degradation Experiment on Rural Domestic Sewage

(1) In the Tanjiawan area of Shiyan, Hubei Province (China), the domestic sewage of the rural septic tank was subjected to grid filtration and sedimentation, and 20 L of the supernatant was added to an integrated sewage treatment apparatus. Test results: the pH of wastewater was 7.2, the ammoniacal nitrogen concentration was 60 mg/L, CODcr was 300 mg/L, and LAS was 5 mg/L.

(2) The liquid containing the LAS-degrading and/or N-removing bacterium of the present invention was directly added to the integrated sewage treatment apparatus containing the 20 L of rural domestic sewage, in an inoculum amount of 10% by volume. The culturing temperature was 30° C. The pH of the target water did not need to be controlled during the sewage decontamination process, and aeration was performed for 1 hour every day. Results: after 2 days of treatment, the removal of ammoniacal nitrogen from the water can reach 88%, the removal of CODcr can reach 60%, and the degradation of LAS was 97%.

The above examples are merely illustration of the present invention and are not intended to limit the scope of protection of the present invention. Any designs identical or similar to the present invention are within the scope of protection of the present invention.

1. A LAS-degrading and/or N-removing bacterium, taxonomically classified as Pseudomonas sp., deposited in China General Microbiological Culture Collection Center on Sep. 18, 2018, with the accession number of CGMCC 16502.

2. The bacterium according to claim 1, wherein the 16S rDNA sequence of the strain of the bacterium is SEQ ID NO: 1.

3. The bacterium according to claim 1, wherein the strain of the bacterium has the following phenotypic characteristics:
when cultured at 36° C. for 24 h, the bacterial colonies are light yellow, circular and opaque, and have a wet surface and a smooth margin; and
the bacterial cells are rod-shaped, 0.4˜0.8 μm×1.8˜4.7 μm in size, arranged individually or in pairs, and gram-negative.

4. The bacterium according to claim 1, wherein the strain of the bacterium has the following characteristics:
(G+C) mol % is 63.4%; and
the quinone components are ubiquinone Q8 and ubiquinone Q9, and their relative contents are 57.24% and 42.76%, respectively.

5. The bacterium according to claim 1, wherein the strain of the bacterium has the following characteristics: the major fatty acid components are: C_{16.0}, Summed Feature 3 (C_{16:1}ω7c or C_{16.1}ω6c), and Summed Feature 8 (C_{18:1}ω7c or C_{18.1}ω6c), and their relative contents are 31.16%, 14.85% and 12.32%, respectively.

6. The bacterium according to claim 1, wherein the strain of the bacterium has the following characteristics: the cell wall-characteristic DAP component is meso-DPA; and the cell wall-characteristic sugar component is ribose.

7. The bacterium according to claim 1, wherein the strain of the bacterium has the following characteristics: the bacterial polar lipids comprise diphosphatidylglycerol, phosphatidylglycerol, phosphatidylethanolamine, phosphatidylcholine, an unidentified phospholipid, an unidentified lipid and an unidentified aminophospholipid.

8. The bacterium according to claim 1, wherein the bacterium shows its LAS-degrading effect over an initial LAS concentration of 0-400 mg/L; and the bacterium shows its ammoniacal nitrogen-removing effect over an initial ammoniacal nitrogen concentration of 0-200 mg/L.

9. The bacterium according to claim 1, wherein the bacterium simultaneously degrades LAS and removes N.

10. Use of the bacterium according to claim 1 for decontamination of rural domestic sewage, wherein the rural domestic sewage has a LAS concentration of 0 to 400 mg/L and/or an ammoniacal nitrogen concentration of 0 to 200 mg/L.

11. The bacterium according to claim 8, wherein the bacterium shows its LAS-degrading effect over an initial LAS concentration of 0-200 mg/L; and the bacterium shows its ammoniacal nitrogen-removing effect over an initial ammoniacal nitrogen concentration of 0-100 mg/L.

12. A method of decontaminating rural domestic sewage, wherein the rural domestic sewage has a LAS concentration of 0 to 400 mg/L and/or an ammoniacal nitrogen concentration of 0 to 200 mg/L, said method comprising adding an effective inoculum amount of the bacterium of claim 1 to the rural domestic sewage.
